(19) United States
(12) Patent Application Publication
(10) Pub. No.: US 2018/0127702 A1
(43) Pub. Date: May 10, 2018

(54) CELL CULTURE VESSEL PACKAGING BAG AND PACKAGE

(71) Applicant: SUMITOMO BAKELITE CO., LTD., Tokyo (JP)

(72) Inventors: Ryohei Tsukada, Tokyo (JP); Aiko Mizuno, Tokyo (JP)

(73) Assignee: SUMITOMO BAKELITE CO., LTD., Tokyo (JP)

(21) Appl. No.: 15/800,448

(22) Filed: Nov. 1, 2017

(30) Foreign Application Priority Data
Nov. 8, 2016 (JP) 2016-218303

(51) Int. Cl. C12M 1/12

(52) U.S. Cl. CPC C12M 37/00

(57) ABSTRACT

It is an object of the invention to provide a cell culture vessel packaging bag that is capable of achieving both the reduction of the amount of adsorption of hydrogen peroxide to a cell culture vessel and the suppression of adverse effects of an oxidizing gas on cells or cell aggregates. Provided is a cell culture vessel packaging bag for including a cell culture vessel, the packaging bag being formed from two or more sheets of film arranged along the direction of gas permeation, in which the film is a laminated film including at least a base material layer in which at least one kind of micropores selected from through-holes and non-through-holes are formed, and a sealant layer, and the micropores are slit-shaped pores and are formed at a density of 1,000 to 10,000 per cm² of the base material layer.

### TECHNICAL FIELD OF THE INVENTION

The present invention relates to a cell culture vessel packaging bag for including a cell culture vessel, and a package obtained by packaging a cell culture vessel inside the cell culture vessel packaging bag.

### RELATED ART

The operation of culturing stem cells such as human embryonic stem cells (human ES cells) and human induced pluripotent stem cells (human iPS cells) or cell aggregates is carried out in a highly aseptic environment. For example, after a cell culture vessel packaged using a packaging bag is introduced into an isolator, any contamination sources such as microorganisms incorporated into the isolator along with the introduction of the cell culture vessel into the isolator are decontaminated by means of a treatment agent such as hydrogen peroxide gas. Subsequently, aeration is performed, and after the hydrogen peroxide concentration inside the isolator reaches a predetermined value or less, the cell culture vessel is taken out from the packaging bag.

Patent Document 1 discloses an example of the cell culture vessel used for the culture of ES cells or iPS cells.

### PRIOR ART DOCUMENT

### Patent Document

[Patent Document 1] Japanese Unexamined Patent Application, First Publication No. 2015-065942

### DISCLOSURE OF THE INVENTION

### Problems to be Solved by the Invention

However, there have been occasions in which the hydrogen peroxide that has permeated through the wrapping material that wraps the cell culture vessel and adsorbed to the cell culture vessel during decontamination, dissolves in the medium in the cell culture vessel, and this hydrogen peroxide in the medium adversely affects the growth of cells or cell aggregates.

Furthermore, there have been occasions in which particularly in a case where a packaging bag formed using a wrapping material having high gas barrier properties is used for the packaging of the cell culture vessel in order to prevent adsorption of hydrogen peroxide to the cell culture vessel, the oxidizing gas generated from the packaging bag and the cell culture vessel by performing γ-ray sterilization adversely affects the growth of cells or cell aggregates.

Thus, it is an object of the present invention to provide a cell culture vessel packaging bag, the packaging bag capable of achieving both the reduction of the amount of adsorption of hydrogen peroxide to a cell culture vessel and the suppression of adverse effects of an oxidizing gas on cells or cell aggregates. It is another object of the present invention to provide a package including a cell culture vessel packaging bag; and a cell culture vessel encapsulated in the cell culture vessel packaging bag, the package causing a reduced amount of hydrogen peroxide to adsorb to the cell culture vessel and enabling satisfactory growth of cells or cell aggregates.

### Means for Solving the Problem

In order to achieve the objects described above, the present invention provides the following cell culture vessel packaging bag and package.

A cell culture vessel packaging bag for including a cell culture vessel,

the cell culture vessel packaging bag being formed from two or more sheets of film arranged along the direction of gas permeation,

in which the film is a laminated film including at least a base material layer having at least one kind of micropores selected from through-holes and non-through-holes; and a sealant layer, and

the micropores are slit-shaped and are formed at a density of 1,000 to 10,000 per cm² of the base material layer.

A package including the cell culture vessel packaging bag; and a cell culture vessel encapsulated in the cell culture vessel packaging bag.

According to the present invention, a cell culture vessel packaging bag that is capable of achieving both the reduction of the amount of adsorption of hydrogen peroxide to a cell culture vessel and the suppression of adverse effects of an oxidizing gas on cells or cell aggregates, can be provided. Furthermore, according to the present invention, a package that causes a reduced amount of hydrogen peroxide to adsorb to a cell culture vessel and enables satisfactory growth of cells or cell aggregates, can be provided.

### BRIEF DESCRIPTION OF THE DRAWINGS

FIG. 1 is a schematic plan view illustrating an example of the cell culture vessel packaging bag of the invention and an example of the package of the invention.

FIG. 2 is a conceptual cross-sectional view illustrating another example of the cell culture vessel packaging bag of the invention and another example of the package of the invention.

FIG. 3 is an expanded conceptual cross-sectional view illustrating an example of a film used for forming the cell culture vessel packaging bag of the invention.

FIG. 4 is a schematic view of a graph obtained by scanning a base material layer that constitutes a laminated film, in the thickness direction using a 3D color laser microscope.

### EMBODIMENTS OF THE INVENTION

According to the present invention, a “non-through-hole” means that the thickness of the thinnest part of the base material layer in the non-through-hole is 70% or less of the thickness at a site where neither a through-hole nor a non-through-hole is formed in the base material layer.

The cell culture vessel packaging bag (hereinafter, may be simply referred to as “packaging bag”) of the invention is a packaging bag for encapsulating a cell culture vessel (hereinafter, may be simply referred to as “culture vessel”), in which two or more sheets of film are arranged along the direction of gas permeation. The film is a laminated film including a base material layer having at least one kind of micropores selected from through-holes and non-through-holes formed therein, and a sealant layer, and the micropores are slit-shaped and are formed at a density of 1,000 to 10,000 per cm² of the base material layer.

The package of the invention can suppress, irrespective of the fact that the package uses a laminated film including a base material layer having micropores formed therein, the adsorption of hydrogen peroxide to the same extent as in the case of using a laminated film including a base material layer having no micropores formed therein, and can also suppress any adverse effects on cells or cell aggregates caused by an oxidizing gas that can be generated in a case where the package is subjected to γ-ray sterilization.

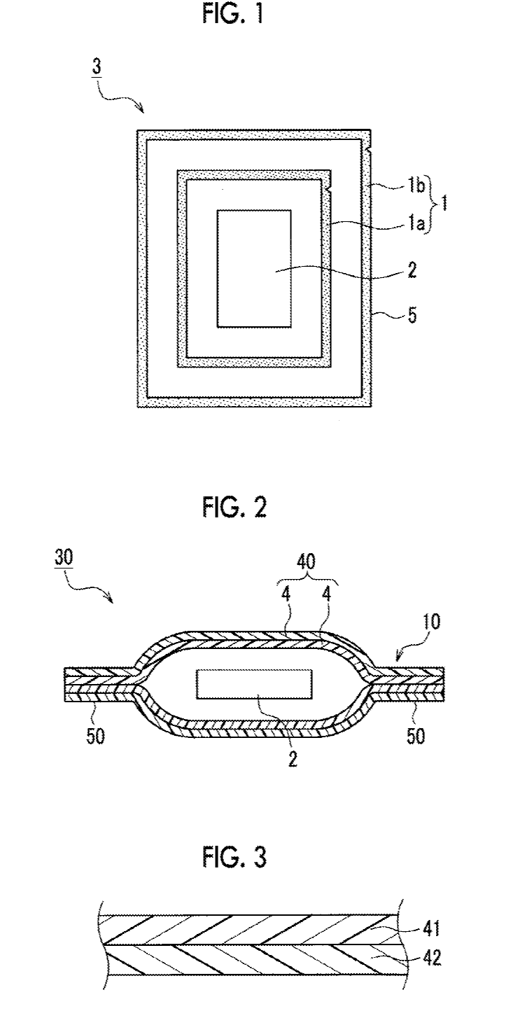

Next, an example of the packaging bag of the invention, an example of a package obtained by packaging a cell culture vessel inside the packaging bag, and an example of the method for producing the package will be explained using FIG. 1 to FIG. 4.

[Laminated Film]

The laminated film used in the packaging bag of the invention (hereinafter, may be referred to as “wrapping material”) includes, for example, as illustrated in FIG. 3, a base material layer 41 having the micropores formed therein; and a sealant layer 42 disposed in contact with one surface of the base material layer 41.

From the viewpoint of maintaining strength, the thickness of the wrapping material is preferably 10 μm or more, and more preferably 30 μm or more, and from the viewpoint of bending resistance, the thickness is preferably 150 μm or less, and more preferably 120 μm or less.

The number of the micropores in the base material layer 41 that constitutes the wrapping material is 1,000 to 10,000, preferably 1,500 to 6,000, and more preferably 2,000 to 5,000, per cm² of the base material layer, for the purpose of securing transpiration of the oxidizing gas and the film strength. Meanwhile, the method for measuring the number of micropores is as described in the Examples.

Specific examples of the base material layer 41 that constitutes the wrapping material include resin films produced from polyolefins such as polyethylene and polypropylene, polyesters such as polyethylene terephthalate and polyethylene-2,6-naphthalate; polyamides such as nylon 6, nylon 66, and nylon 12; polyimides, polyetherimides, polycarbonates (PC), polyvinyl butyral, and fluororesins as raw materials. Among these, from the viewpoints of having high transparency and low moisture absorption, a polyester film is more preferred, and a polyethylene terephthalate film is even more preferred. From the viewpoint of high flexibility, a polyolefin film is preferred. From the viewpoints of suppressing adsorption of hydrogen peroxide and securing impact resistance, a polyamide film is preferred. Furthermore, the base material layer 41 may be a multilayer film in which films produced from the above-mentioned resins as raw materials are laminated; however, from the viewpoint of the ease of production, the base material layer 41 is preferably a single-layer film, and more preferably a single-layer polyamide film.

The base material layer 41 may be any of an unstretched film, a uniaxially stretched film, and a biaxially stretched film; however, from the viewpoint of having high barrier properties, the base material layer 41 is preferably a uniaxially stretched film or a biaxially stretched film, and more preferably a biaxially stretched film.

From the viewpoint of achieving both the operability of unsealing and the securement of mechanical strength of the packaging bag, the thickness of the base material layer 41 is preferably 3 to 150 μm, more preferably 5 to 120 μm, even more preferably 10 to 100 μm, and even still more preferably 10 to 50 μm.

The shape of the micropores formed in the base material layer 41 is slit-shaped, from the viewpoint of achieving both the securement of hydrogen peroxide barrier properties and the enhancement of transpirability for an oxidizing gas. Examples of slit-shaped micropores include linear incisions, cracks, and crevices. Examples of the method for forming micropores include general methods that are conventionally known, for example, the methods disclosed in Japanese Unexamined Patent Application, First Publication No. H07-256807 and Japanese Unexamined Patent Application, First Publication No. H07-164535; however, the micropores may be formed using any means such as heat, laser light, ultrasonic waves, and electricity.

The average upper side length of the micropores is preferably 10 to 200 μm, more preferably 15 to 180 μm, even more preferably 20 to 150 μm, and still more preferably 40 to 70 μm, from the viewpoint of achieving both the securement of hydrogen peroxide barrier properties and the enhancement of transpirability for an oxidizing gas.

The upper side length of a micropore is the length of the micropore in the principal surface of the base material layer that constitutes one of the principal surfaces of the wrapping material. The upper side length can be read out by the method described in the Examples from the data obtained by scanning the base material layer in the thickness direction using a 3D color laser microscope.

The average lower side length of the micropores is preferably 1 to 100 μm, more preferably 1.5 to 90 μm, even more preferably 2 to 80 μm, and still more preferably 10 to 30 μm, from the viewpoint of achieving both the securement of hydrogen peroxide barrier properties and the enhancement of transpirability for an oxidizing gas.

The lower side length of a micropore is the length of the micropore at the surface on the opposite side of the principal surface where the “upper side length” is measured, between the both ends of the base material layer in the thickness direction. The lower side length can be read out by the method described in the Examples from the data obtained by scanning the base material layer in the thickness direction using a 3D color laser microscope.

The lower side length of the micropores is smaller than the upper side length, and the average lower side length is also smaller than the average upper side length.

The sealant layer 42 is the outermost layer of one side of the laminated film, and is a heat-sealable resin layer that is melted by heat. The sealant layer is also referred to as heat seal layer. Specific examples of the sealant layer 42 include resin films of polyethylene, polypropylene, an ethylene-vinyl acetate copolymer, an ethylene-acrylic acid copolymer, an ethylene-methacrylic acid copolymer, and the like. Regarding the method for laminating the sealant layer 42 on the base material layer 41, a known extrusion lamination method, a dry lamination method, and the like are employed. Furthermore, the sealant layer 42 may be a multilayer film in which films produced from the above-mentioned resins as raw materials are laminated; however, from the viewpoint of the ease of production, the sealant layer 42 is preferably a single-layer film, and more preferably a single-layer polyethylene film.

From the viewpoint of achieving both the securement of hydrogen peroxide barrier properties and the enhancement of transpirability for an oxidizing gas, the thickness of the sealant layer 42 is preferably 10 to 100 μm, more preferably 15 to 90 μm, even more preferably 20 to 80 μm, and still more preferably 50 to 60 μm.

From the viewpoint of achieving both the securement of hydrogen peroxide barrier properties and the enhancement of transpirability for an oxidizing gas, a preferred layer configuration of the wrapping material is preferably heat-sealable polyolefin resin film/polyester film (sealant layer/base material layer), or heat-sealable polyolefin resin film/polyamide film/polyolefin resin film (sealant layer/base material layer/base material layer); more preferably polyethylene film/polyethylene terephthalate film (sealant layer/base material layer), or polyethylene film/polyamide film (sealant layer/base material layer); even more preferably polyethylene film/polyamide film (sealant layer/base material layer); and still more preferably polyethylene film/nylon film (sealant layer/base material layer).

In a case where the package of the invention produced by encapsulating a culture vessel in the packaging bag is treated under the conditions described below, the amount of hydrogen peroxide that adsorbs to the culture vessel can be preferably less than 30 ng/cm², more preferably less than 20 ng/cm², and even more preferably less than 10 ng/cm², and thus the adverse effects on the productivity of cells or cell aggregates caused by hydrogen peroxide can be effectively suppressed.

The treatment conditions for the package are as follows.

In a tightly sealed space having a volume of 170 L, which has been maintained at normal pressure and a temperature of 45° C., a packaging bag having a culture vessel encapsulated therein (that is, package of the invention) is placed, 70 mL of a 6 mass % aqueous solution of hydrogen peroxide is vaporized by applying ultrasonic vibration to produce hydrogen peroxide vapor. Then, the packaging bag is exposed to this hydrogen peroxide vapor for 7 minutes. Within 90 minutes from the termination of ultrasonic vibration, hydrogen peroxide is decomposed by irradiating with ultraviolet radiation having a wavelength of 254 nm, such that the hydrogen peroxide concentration in the tightly sealed space will become 1 mg/L or less. Normal pressure means a pressure equal to the atmospheric pressure, and this is almost 1 atmosphere.

According to the present invention, the “amount of hydrogen peroxide (ng/cm²) adsorbing to the cell culture vessel” is obtained by taking out the culture vessel from the packaging bag that has been subjected to the treatment described above, rapidly dissolving the hydrogen peroxide that has adsorbed to the culture vessel in ultrapure water, measuring the concentration of hydrogen peroxide in the solution, and converting this concentration to the amount of adsorption per unit area. The concentration of hydrogen peroxide in the solution is determined from the absorbance at 550 nm of the solution to which an enzyme and a color developer have been added, and a calibration curve that has been produced in advance.

Examples of the enzyme include the peroxidases described in the Examples that will be described below. Examples of the color developer include 4-aminoantipyrine described in the Examples described below. The details of the method for calculating the “amount of adsorption of hydrogen peroxide (ng/cm) to the cell culture vessel” are as described in the Examples.

The packaging bag of the invention may be a multiple packaging bag including an inner bag formed from the aforementioned wrapping material; and one or more outer bags formed from the wrapping material. The multiple packaging beg may be, for example, a double packaging bag 1 that includes an inner bag 1a and an outer bag 1b as illustrated in FIG. 1 so that the culture vessel 2 is covered with a double wrapping material; may be a triple packaging bag including two or more outer bags 1b so that the culture vessel 2 is covered with a triple wrapping material; or may be a quadruple packaging bag with the culture vessel being covered with a quadruple or higher-order wrapping material. From the viewpoints of the operability of unsealing, and suppression of the generation of dust at the time of operation of unsealing, quintuple or fewer are preferred. The inner bag 1a and the outer bag 1b are each a soft packaging bag produced by providing a heat sealing portion 5 on two sides or three sides along the periphery of a wrapping material that has been folded into a half or has been formed by overlapping two sheets, and forming an opening on one side.

As illustrated in FIG. 2, the packaging bag may also be a packaging bag 10 formed using a multiple film 40, in which two or more sheets of the wrapping material 4 are overlapped but are not joined to each other. In this case, the packaging bag 10 is, for example, a soft packaging bag produced by providing a heat sealing portion 50 on two sides or three sides along the periphery of a multiple film 40 that has been folded into a half or has been formed by overlapping, and forming an opening on one side. The respective films 4 in the multiple film 40 at the parts other than the parts where the heat sealing portion 50 is formed are not joined to each other and are only in contact with each other.

Packages 3 and 30 including a packaging bag and a culture vessel encapsulated in the packaging bag are obtained by inserting a culture vessel 2 into packaging bags 1 and 10 (see FIG. 1 and FIG. 2) and heat-sealing the openings. In a case where the packaging bag of the invention is a multiple packaging bag, an example of the package of the invention (see, for example, FIG. 1) is obtained by inserting a culture vessel into the innermost bag that constitutes the multiple packaging bag; heat-sealing the opening of that bag to encapsulate the culture vessel inside that bag; subsequently encapsulating the bag in which the culture vessel is encapsulated, inside another bag that constitutes the multiple packaging bag; and repeating this operation as many times as the number of the remaining bags that constitute the multiple packaging bag. The packaging bag of the invention and the respective bags that constitute the packaging bag of the invention may respectively include a nick for unsealing, such as a V-notch.

There are no particular limitations on the form of the culture vessel that is encapsulated inside the packaging bag of the invention or the culture vessel that constitutes the package of the invention; however, for example, a Petri dish and a culture vessel having a plurality of wells may be mentioned. The packaging bag of the invention is suitable for the packaging of, among various culture vessels, those culture vessels for culturing stem cells such as human embryonic stem cells (human ES cells) or human induced pluripotent stem cells (human iPS cells), or cell aggregates thereof which are required to undergo long-term cell culture and are likely to be adversely affected by hydrogen peroxide.

Furthermore, the shapes of the packaging bag of the invention and the respective bags that constitute the packaging bag of the invention, which have been explained using FIG. 1 and FIG. 2, are respectively flat bag shapes; however, a bag with gusset may also be used.

The packaging bag of the invention is subjected to radioactive sterilization, and may also be subjected not only to γ-ray sterilization but also to electron beam sterilization.

[Sterilization]

The package of the invention may be subjected to radioactive sterilization such as γ-ray sterilization.

The dose rate of radiation in the case of performing radioactive sterilization is 0.1 kGy/hr or more, preferably 1 kGy/hr or more, more preferably 10 kGy/hr or more, and even more preferably 100 kGy/hr or more, from the viewpoint of suppressing the amount of the oxidizing gas generated from the packaging bag and the culture vessel. From the viewpoint of suppressing heat generation, the dose rate is preferably 100 MGy/hr or less, preferably 10 MGy/hr or less, and more preferably 1 MGy/hr (100 kGy/hr) or less. In electron beam sterilization, generally, the dose rate of radiation is preferably from 100 kGy/hr to 10 MGy/hr, and in γ-ray sterilization, generally, the dose rate of radiation is preferably from 0.1 kGy/hr to 10 kGy/hr.

The exposure dose of radiation is determined by any of the methods described in JIS-T 0806 depending on the level of sterility required for packages. Meanwhile, the exposure dose of radiation means the cumulative radiation absorbed dose of the package.

The material of the culture vessel is not particularly limited; however, from the viewpoint that the culture vessel can be made into a disposable type and easy molding is allowed, a resin is preferred. Examples of the resin include polyolefin-based resins or cyclic polyolefin-based resins, such as a polypropylene resin, a polyethylene resin, and an ethylene-propylene copolymer; polystyrene-based resins such as polystyrene and an acrylonitrile-butadiene-styrene-based resin; methacrylic-based resins such as a polycarbonate resin, a polyethylene terephthalate resin, and a polymethyl methacylate resin; a vinyl chloride resin, a polybutylene terephthalate resin, a polyallylate resin, a polysulfone resin, a polyether sulfone resin, a polyether ether ketone resin, a polyetherimide resin; fluoro-based resins such as a polytetrafluoroethylene resin, a polymethylpentene resin; acrylic resins such as polyacrylonitrile; and cellulosic-based resins such as propionate resin. Among these, a polystyrene resin is preferred from the viewpoints of the moldability and sterility required for culture vessels.

There are no particular limitations on the shape of the culture vessel, and examples thereof include a Petri dish (dish), a multi-well plate including a plurality of wells, a flask, and a tube. However, among these, a multi-well plate and a Petri dish, which are used for the production of bioreactors, assessment of efficacy or toxicity, development and research of artificial organs, and the like. A multi-well plate is a substrate having a plurality of concavities opened at the top face. The number of wells in a multi-well plate is not particularly limited; however, the number of wells is, for example, 6, 12, 24, 48, 96, or 384. Examples of the multi-well plate include those disclosed in Japanese Unexamined Patent Application, First Publication No. 2013-70636 and Japanese Unexamined Patent Application, First Publication No. 2012-210166.

[Decontamination Method]

It is preferable that the package of the invention is decontaminated. The method for decontamination may be any of a method of immersing the package of the invention in hydrogen peroxide, a method of exposing the package of the invention to hydrogen peroxide plasma or hydrogen peroxide water vapor, and the like. However, the exposure to hydrogen peroxide plasma or hydrogen peroxide water vapor is preferred, by which decontamination is carried out effectively and the culture vessel is less adversely affected.

In a case where the package of the invention is decontaminated using hydrogen peroxide plasma or hydrogen peroxide water vapor, for example, decontamination can be carried out as follows.

The package of the invention is introduced into an isolator and is exposed to hydrogen peroxide plasma or hydrogen peroxide water vapor, and then the concentration of hydrogen peroxide plasma or hydrogen peroxide water vapor in the isolator is decreased (decontamination process).

More specifically, for example, the package of the invention is introduced into an isolator, subsequently hydrogen peroxide is supplied into the isolator, and the package is exposed to an atmosphere in which the concentration of hydrogen peroxide plasma or hydrogen peroxide water vapor is preferably 10 to 1,000 ppm, preferably for 1 to 60 minutes. Subsequently, preferably within 150 minutes, the hydrogen peroxide is discharged while clean air is introduced into the isolator from the outside such that the concentration of hydrogen peroxide inside the isolator is preferably 1 ppm or less, or the hydrogen peroxide inside the isolator is decomposed by ultraviolet irradiation.

The present invention further relates to one or a plurality of embodiments described below.

[1] A cell culture vessel packaging bag for including a cell culture vessel,

the cell culture vessel packaging bag being formed of two or more sheets of film arranged along the direction of gas permeation,

wherein the film is a laminated film including at least a base material layer in which at least one kind of micropores selected from through-boles and non-through-holes are formed; and a sealant layer, and the micropores are slit-shaped and are formed at a density of 1,000 to 10,000 per cm² of the base material layer.

[2] The cell culture vessel packaging bag according to [1], wherein the average upper side length of the micropores obtained by scanning the base material layer in the thickness direction using a 3D color laser microscope, is 10 to 200 μm.

[3] The cell culture vessel packaging bag according to [1] or [2], wherein the average lower side length of the micropores obtained by scanning the base material layer in the thickness direction using a 3D color laser microscope is, 1 to 100 μm.

[4] The cell culture vessel packaging bag according to any one of [1] to [3], wherein the amount of adsorption of hydrogen peroxide of the cell culture vessel in a case where the cell culture vessel packaging bag having the cell culture vessel encapsulated therein is treated under the following conditions, is less than 30 ng/cm²:

treatment conditions:

in a tightly sealed space having a volume of 170 L, which has been maintained at normal pressure and at a temperature of 45° C., the cell culture vessel packaging bag having a cell culture vessel encapsulated therein is introduced, 70 mL of a 6 mass % aqueous solution of hydrogen peroxide is vaporized by applying ultrasonic vibration to produce hydrogen peroxide water vapor, the packaging bag is exposed to this hydrogen peroxide vapor for 7 minutes, and the hydrogen peroxide is decomposed by irradiating with ultraviolet radiation having a wavelength of 254 nm within 90 minutes from the termination of ultraviolet vibration, such that the hydrogen peroxide concentration inside the tightly sealed space becomes 1 mg/L or less.

[5] The cell culture vessel packaging bag according to any one of [I] to [4], wherein the material of the base material layer is a polyester or a polyamide.

[6] The cell culture vessel packaging bag according to any one of [1] to [5], wherein the cell culture vessel packaging bag is a multiple package including an inner bag formed from the above-mentioned film, and one or more outer bags formed from the above-mentioned film, and there is a space provided between the inner bag and the outer bag.

[7] The cell culture vessel packaging bag according to any one of [1] to [5], wherein the cell culture vessel packaging bag is a packaging bag formed using a multiple film obtained by overlapping two or more sheets of the above-mentioned film.

[8] A package, including the cell culture vessel packaging bag according to any one of [l] to [7]; and a cell culture vessel encapsulated inside the cell culture vessel packaging bag.

[9] A method for producing a package, the method including:

a step of encapsulating a cell culture vessel inside the cell culture vessel packaging bag according to any one of [1] to [7]; and

a sterilization step of subjecting the cell culture vessel packaging bag having the cell culture vessel encapsulated therein, to γ-ray sterilization.

[10] The method for producing a package according to [9], wherein in the sterilization step, the cell culture vessel packaging bag having the cell culture vessel encapsulated therein is irradiated with γ-radiation at a dose of from 0.1 kGy/hr to 10 kGy/hr.

[11] A method for decontaminating a package, the method including a step of performing a hydrogen peroxide treatment for a package produced by the method for producing a package according to [9] or [10],

wherein in the step of performing a hydrogen peroxide treatment,

after the package is exposed to hydrogen peroxide, the hydrogen peroxide concentration in the atmosphere including hydrogen peroxide is reduced.

[12] The method for decontaminating a package according to [11], wherein in the step of performing a hydrogen peroxide treatment,

the package is exposed to hydrogen peroxide water vapor or hydrogen peroxide plasma.

[13] The method for decontaminating a package according to [11] or [12], wherein in the step of performing a hydrogen peroxide treatment,

the hydrogen peroxide concentration in the atmosphere is reduced by irradiating the hydrogen peroxide water vapor with ultraviolet radiation to decompose hydrogen peroxide.

[14] The method for decontaminating a package according to [11] or [12], wherein in the step of performing a hydrogen peroxide treatment,

the hydrogen peroxide concentration in the atmosphere is reduced by discharging hydrogen peroxide from the atmosphere.

Hereinafter, the present invention will be explained based on the following Examples and Comparative Examples; however, the present invention is not intended to be limited to these.

### EXAMPLES

[Number of Micropores Formed in Base Material Layer]

A base material layer was observed with a 3D color laser microscope (manufactured by Keyence Corporation, model VK-9700/9710), and the number of micropores per 0.015 square centimeters (1012 μm in length×1350 μm in width) was counted at any arbitrary 10 sites. The number of micropores per 1 square centimeter was calculated from these values, and the average value is presented in the following Table 1.

[Average Upper Side Length and Average Lower Side Length of Micropores]

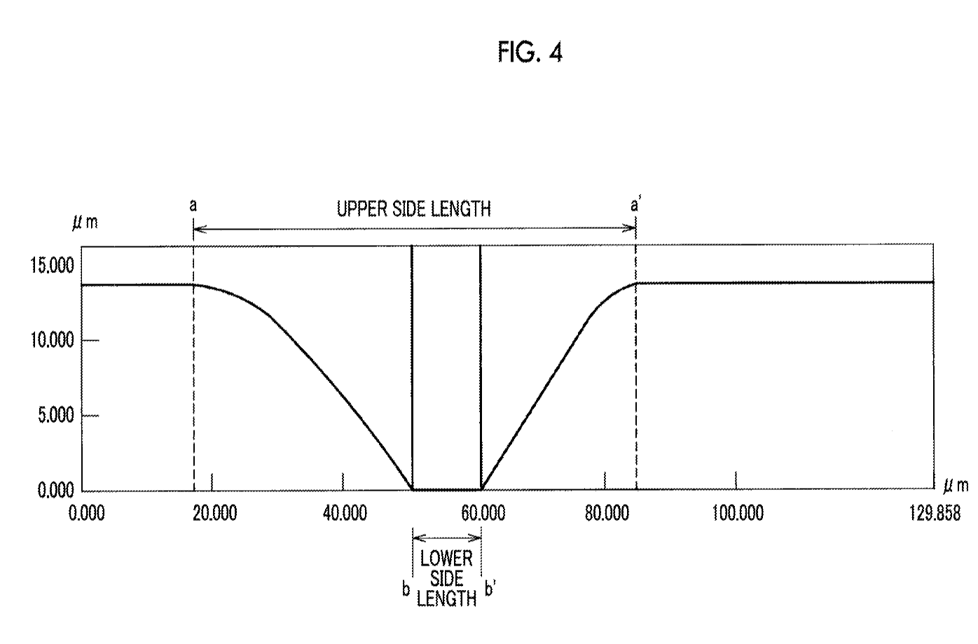

From the data obtained by scanning a base material layer in the Z-axis direction (thickness direction of the base material layer) using a 3D color laser microscope (manufactured by Keyence Corporation, model VK-9700/9710), the “upper side length” and the “lower side length” were measured for 35 micropores. From the information of the difference in height obtained by performing profile measurement on a straight line along each micropore, as illustrated in the schematic view of FIG. 4, the horizontal distance between points a and a′, where the height begins to lower compared to the vicinity, was designated as “upper side length”, and the length of the bottom of the pore (length between points b and b′ of the part that is particularly low compared to the vicinity) was designated as “lower side length”. The average values are presented in Table 1. The “straight line along a micropore” is a straight line including the longest straight line that can be drawn from a region having the darkest color in a color CCD image of the XY plane of the base material layer.

TABLE 1

| | |
|---|---|
| Average value of number of micropores [micropores/cm²] | 3308 |
| Average upper side length of micropores [μm] | 67 |
| Average lower side length of micropores [μm] | 25 |

[Hydrogen Peroxide Permeation Test]

(1) A Petri dish made of a polystyrene resin and having a diameter of 90 mm (MS-13900, manufactured by Sumitomo Bakelite Co., Ltd.) was encapsulated inside each of the packaging bags of Examples 1 and 2, Comparative Example 1, and Experimental Example 1 as described below, and thus Petri dish-including packaging bags (packages) were obtained.

(2) An H₂O₂ generator (MCO-HP-PJ, manufactured by Panasonic Corporation) was connected to the interior of a CO₂ incubator (MCO-170AICUVH-PJ, volume 170 L, manufactured by Panasonic Corporation). Then, the packages obtained in (I) were introduced into the CO₂ incubator, and then 78 mL of hydrogen peroxide water (6 wt % hydrogen peroxide water, manufactured by Panasonic Corporation) was supplied to the H₂O₂ generator. Subsequently, the temperature inside the incubator was raised to 45° C. Then, hydrogen peroxide vapor was generated by means of ultrasonic waves, and the hydrogen peroxide vapor was sprayed to the packages in an atmosphere at normal pressure and 45° C. The time for applying ultrasonic waves to the hydrogen peroxide water was 7 minutes. After termination of the application of ultrasonic waves to the hydrogen peroxide water, the hydrogen peroxide water vapor was immediately irradiated with ultraviolet radiation having a wavelength of 254 nm for 90 minutes to thereby decompose hydrogen peroxide, such that the hydrogen peroxide water vapor concentration inside the CO₂ incubator was 1 mg/L or less.

The procedure from the temperature rise to 45° C. to the decomposition of hydrogen peroxide was carried out by the programs of the instruments used in the present Example. Since 8 mL of hydrogen peroxide water remained inside the CO₂ incubator after the irradiation with ultraviolet radiation, it was considered that 70 ml of hydrogen peroxide water was used for the spraying of hydrogen peroxide vapor.

(3) Subsequently, the Petri dish was removed from the packaging bag, 3 ml of ultrapure water was introduced into the Petri dish that was placed on a horizontal surface, and the hydrogen peroxide that had adsorbed to the bottom face of the Petri dish and parts of the inner side surface of the Petri dish adjacent to the bottom face (hereinafter, referred to as “surface to be cleaned”) was dissolved in that ultrapure water. The area of the surface to be cleaned was 58.5 cm².

(4) The ultrapure water containing dissolved hydrogen peroxide, which was obtained in (3), was subjected to color development with PACKTEST hydrogen peroxide (manufactured by Kyoritsu Chemical-Check Laboratory Corporation, WAK-H₂O₂, measurement range H₂O₂ 0.05 to 5 ppm, 4-aminoantipyrine method using peroxidase), and the absorbance of light having a wavelength of 550 nm was measured. The absorbance thus obtained was converted to the residual hydrogen peroxide concentration [ppm] and the amount of adsorption of hydrogen peroxide [ng/cm²] using a separately produced calibration curve. These values are presented in Table 2.

[Colony Formation Test]

The packages of Example 3 and Comparative Example 2 as described below, which were subjected to γ-ray sterilization, were left to stand for 4 weeks at room temperature (25° C.). Subsequently, one NS-1 (mouse myeloma) cell and 0.1 ml of a 10% fetal bovine serum-added MEM medium were placed in each well, and the cells were cultured for 7 days in an atmosphere at 37° C. and 5% CO₂. Subsequently, the number of wells in which the NS-1 cell proliferated and formed colonies was examined, and the results are presented in Table 2.

The details of the packages, packaging bags, and the wrapping materials obtained in Examples 1 to 3, Comparative Examples 1 and 2, and Experimental Example 1 are as follows.

### Example 1

An inner bag having a size of 145 mm×220 mm and an outer bag having a size of 176 mm×380 mm were produced using a wrapping material obtained by laminating a polyethylene sheet (thickness 60 μm) as a heat-sealing layer on one surface of a nylon film having micropores formed therein (thickness 15 μm; see Table 1 for the number of micropores, the average upper side length and the average lower side length of micropores). One Petri dish made of a polystyrene resin and having a diameter of 90 mm (MS-13900, manufactured by Sumitomo Bakelite Co., Ltd.) was inserted into the inner bag, and the inner bag was heat-sealed. Subsequently, the inner bag was inserted into the outer bag, and the outer bag was heat-sealed. Thus, a package (double packaging) of Example 1 was produced.

### Example 2

The package of Example 1 was further inserted into an outer bag having a size of 176 mm×380 mm, and the outer bag was heat-sealed. Thus, a package (triple packaging) of Example 2 was produced.

### Example 3

A package (triple packaging) was produced in the same manner as in Example 2, except that a 96-well plate made of a polystyrene resin (MS-8096F, manufactured by Sumitomo Bakelite Co., Ltd.) was used instead of the Petri dish made of a polystyrene resin and having a diameter of 90 mm. The package thus obtained was irradiated with γ-radiation. In order to adjust the sterility level of the 96-well plate to the sterility level required for medical tools, the dose rate of γ-radiation was set to 1 kGy/hr, and the exposure dose thereof was set to 19 kGy.

### Comparative Example 1

A package (single packaging) of Comparative Example 1 was obtained in the same manner as in Example 1, except that the inner bag including the Petri dish made of a polystyrene resin and having a diameter of 90 mm was not inserted into an outer bag.

### Comparative Example 2

A bag having a size of 145 mm×220 mm was produced using a wrapping material obtained by laminating a polyethylene sheet (thickness 70 μm) as a heat-seal layer on one surface of a nylon film (15 μm) in which no micropores were formed. One 96-well plate made of a polystyrene resin (MS-8096F, manufactured by Sumitomo Bakelite Co., Ltd.) was inserted into the bag, and the bag was heat-sealed. Thus, a package of Comparative Example 2 was produced. The package thus obtained was irradiated with γ-radiation. The dose rate of γ-radiation was set to 1 kGy/hr, and the exposure dose thereof was set to 19 kGy.

### Experimental Example 1

An inner bag having a size of 145 mm×220 mm and an outer bag having a size of 176 mm×380 mm were produced using a wrapping material obtained by laminating a polyethylene sheet (thickness 60 μm) as a heat-seal layer on one surface of a nylon film (15 μm) in which no micropores were formed. One Petri dish made of a polystyrene resin and having a diameter of 90 mm (MS-13900, manufactured by Sumitomo Bakelite Co., Ltd.) was inserted into the inner bag, and the inner bag was heat-sealed. Subsequently, the inner bag was inserted into the outer bag, and the outer bag was heat-sealed. Thus, a package (double packaging) of Experimental Example 1 was obtained.

TABLE 2

| | | | | Comparative | Comparative | Experimental |
|---|---|---|---|---|---|---|
| | Example 1 | Example 2 | Example 3 | Example 1 | Example 2 | Example 1 |
| Residual | 0.185 | 0.012 | — | 5.785 | | 0.17 |
| hydrogen | | | | | | |
| peroxide | | | | | | |
| concentration | | | | | | |
| [ppm] | | | | | | |
| Amount of | 9.74 | 0.64 | — | 304.48 | | 8.95 |
| adsorption of | | | | | | |
| hydrogen | | | | | | |
| peroxide | | | | | | |
| [ng/cm²] | | | | | | |
| Number of | — | — | 44 | — | 0 | — |
| wells with | | | | | | |
| colony formation | | | | | | |
| [wells/96 wells] | | | | | | |

As shown in Table 2, when a comparison was made between Examples 1 and 2 and Comparative Example 1, in Examples 1 and 2, the amount of adsorption of hydrogen peroxide to the culture vessel was extraordinarily small compared to Comparative Example 1. Therefore, in Examples 1 and 2, suppression of adverse effects of hydrogen peroxide on the productivity of cells and cell aggregates can be expected, compared to Comparative Example 1. Furthermore, according to pp. 2-3 of “Heisei Year 25 Report on Industrialization Promotion Project for Regenerative Medicine and the like (Age-related Macular Degeneration, Retinal Pigment Epithelial Cells Derived from Allogeneic iPS cells), it is described that if the dissolved amount of hydrogen peroxide in the medium is more than 0.5 ppm (mg/ml) (when converted to the amount of adsorption of hydrogen peroxide that can be determined by the method described in the above section [Hydrogen peroxide permeation test], 30 ng/cm²), the survival rate of iPS cells is markedly lowered. Therefore, in Examples 1 and 2, it is speculated that the survival rate of iPS cells will be higher than that of Comparative Example 1.

The amount of adsorption of hydrogen peroxide of Example 3 was not measured. However, since the only difference between Example 2 and Example 3 was the type of the cell culture vessel to be accommodated, it is anticipated that the amount of adsorption of hydrogen peroxide of Example 3 will be equivalent to that of Example 2.

It is understood that the amounts of adsorption of hydrogen peroxide of Example 1 and Experimental Example 1 were equivalent, and the presence of micropores did not adversely affect the permeation of hydrogen peroxide. When a comparison was made between Comparative Example 2, which used a film without any micropores formed therein, and Example 3 with regard to the number of wells with colony formation, the number is significantly larger in Example 3 than in Comparative Example 2. Therefore, in Example 3, it is understood that the oxidizing gas generated by radioactive irradiation was transpirable due to the presence of micropores. From this, it is understood that the packaging bags of the invention are capable of achieving both the reduction of the amount of adsorption of hydrogen peroxide to the cell culture vessel and the suppression of adverse effects of an oxidizing gas on cells or cell aggregates.

### INDUSTRIAL APPLICABILITY

The present invention is useful in, for example, the medical fields such as research on human ES cells and regenerative medicine.

The present invention can be carried out in embodiments other than those described above, to the extent that the gist of the invention is maintained. The embodiments disclosed in the present patent application are only examples, and the invention is not intended to be limited to these. The scope of the present invention is to be construed based on the description of the attached claims, in preference to the above description of the specification, and any modifications made within a scope equivalent to the scope of the claims are all intended to be included in the claims.

### BRIEF DESCRIPTION OF THE REFERENCE SYMBOLS

1, 10: CELL CULTURE VESSEL PACKAGING BAG
1a: INNER BAG
1b: OUTER BAG
2: CELL CULTURE VESSEL
3, 30: PACKAGE
4: FILM
5: HEAT SEALING PORTION

1. A cell culture vessel packaging bag for including a cell culture vessel,
the cell culture vessel packaging bag being formed of two or more sheets of film arranged along a direction of gas permeation,
wherein the film is a laminated film including at least a base material layer having at least one kind of micropores selected from through-holes and non-through-holes; and a sealant layer, and
the micropores are slit-shaped pores and are formed at a density of 1,000 to 10,000 per cm² of the base material layer.

2. The cell culture vessel packaging bag according to claim 1, wherein the average upper side length of the micropores obtained by scanning the base material layer in the thickness direction using a 3D color laser microscope, is 10 to 200 μm.

3. The cell culture vessel packaging bag according to claim 1, wherein the average lower side length of the micropores obtained by scanning the base material layer in the thickness direction using a 3D color laser microscope, is 1 to 100 μm.

4. The cell culture vessel packaging bag according to claim 1, wherein the amount of hydrogen peroxide adsorbing to the cell culture vessel in a case where the cell culture vessel packaging bag having the cell culture vessel encapsulated therein is treated under the following conditions, is less than 30 ng/cm²:
treatment conditions:
in a tightly sealed space having a volume of 170 L, which has been maintained at normal pressure and a temperature of 45° C., the cell culture vessel packaging bag having a cell culture vessel encapsulated therein is introduced, 70 mL of a 6 mass % aqueous solution of hydrogen peroxide is vaporized by applying ultrasonic vibration to produce hydrogen peroxide water vapor, the packaging bag is exposed to this hydrogen peroxide vapor for 7 minutes, and the hydrogen peroxide is decomposed by irradiating with ultraviolet radiation having a wavelength of 254 nm within 90 minutes from the termination of ultrasonic vibration, such that the hydrogen peroxide concentration in the tightly sealed space becomes 1 mg/L or less.

5. The cell culture vessel packaging bag according to claim 1, wherein the material for the base material layer is a polyester or a polyamide.

6. The cell culture vessel packaging bag according to claim 1, wherein the cell culture vessel packaging bag is a multiple packaging bag including an inner bag formed of the film, and one or more outer bags formed of the film, and there is a space provided between the inner bag and the outer bag.

7. The cell culture vessel packaging bag according to claim 1, wherein the cell culture vessel packaging bag is a packaging bag formed using a multiple film obtained by overlapping two or more sheets of the film described above.

8. A package comprising:
the cell culture vessel packaging bag according to claim 1; and
a cell culture vessel encapsulated inside the cell culture vessel packaging bag.
